(19) United States
(12) Patent Application Publication
(10) Pub. No.: US 2014/0371840 A1
(43) Pub. Date: Dec. 18, 2014

(54) GRADED COMPLIANT ENDOGRAFT AND METHOD OF CONSTRUCTING THE SAME

(71) Applicants: Ramon Berguer, West Bloomfield, MI (US); Juan Parodi, Buenos Aires (AR)

(72) Inventors: Ramon Berguer, West Bloomfield, MI (US); Juan Parodi, Buenos Aires (AR)

(21) Appl. No.: 14/303,264

(22) Filed: Jun. 12, 2014

Related U.S. Application Data
(60) Provisional application No. 61/834,134, filed on Jun. 12, 2013.

(51) Int. Cl. A61F 2/07; A61F 2/82

(52) U.S. Cl. CPC A61F 2/07; A61F 2/82; A61F 2250/0018; A61F 2250/0039; A61F 2250/0029
USPC 623/1.13

(57) ABSTRACT

An implantable endograft device having a graded compliance. The endograft contains at least two portions having different compliance values. In some configurations, the endograft has three, a proximal end portion, a distal end portion, and a central portion, with the proximal and distal end portions having a higher compliance than the central portion. The difference in compliance can be achieved, for example, by using wire struts of different gauges, varying the spacing of the struts in the different portions, varying the geometry of the struts, using struts of different alloys, or using struts of differing elasticity.

### CROSS REFERENCE TO RELATED APPLICATIONS

The present application claims priority to U.S. Provisional Application No. 61/834,134, filed Jun. 12, 2013, the entire contents of which are incorporated herein by reference.

### FIELD OF THE INVENTION

The present invention relates to a tubular endograft having a graded compliance. In particular, the compliance of at least one end portion of the endograft is different from the compliance of the remainder of the endograft.

### BACKGROUND OF THE INVENTION

Endografts are comprised of stent rings (struts) which serve as a frame for the structure and are attached in some manner to a tubular graft sleeve. Endografts are inserted into an artery and secured in place by the radial force of the struts which anchor them at the site at which they are placed. The insertion method involves the use of a catheter, which introduces the endograft percutaneously into the patient until the endograft either self-expands or is balloon expanded.

A common treatment procedure for aneurysms is the insertion of an endograft. Aneurysms are a ballooning of the blood vessels, which occur as a result of the weakening of blood vessel walls due to degeneration from aging, atherosclerosis, injury and other conditions. If left untreated, aneurysms can rupture, creating a life threatening condition. Endografts are devices that can be implanted within aneurysms to reduce the likelihood of a rupture. Endografts are also used to line the inside of a vessel that was blocked by plaque and has been treated.

Currently available endografts have a constant compliance throughout their structure. Compliance is traditionally defined as C=ΔD/DP_{P}, where D is diameter in diastole, ΔD is the change in diameter and P_{P} is the pulse pressure. One of the main problems that follows the implantation of an endograft inside an artery that has different elastic properties than the endograft is that there are flow disturbances occurring as the flow enters and leaves the endograft. These flow disturbances which occur at both ends of the endograft are a result of the change in geometry, and the abrupt change in compliance between the graft and the native artery. The native artery is a pulsatile, flexible tube which carries a pulse wave with a systolic and diastolic phase. Endografts are more rigid and non-compliant than the arteries within which they are inserted. When the pulse wave of the blood flow reaches the endograft, the pulsatile component of the diastolic recoil is lost, causing a loss of pulsatile energy. As the stream of flow is constricted by the proximal attachment of the more rigid endograft, abnormal vibrations and turbulence at this site cause increased wall stress that stimulates the growth of the inner lining of the native artery. There is also a change in flow patterns at the end of the endograft where the stream of flow enters a more elastic native artery, resulting in turbulence and increased wall stress. This growth of the intima at the proximal and distal attachment sites is called intimal hyperplasia and is akin to a scarring of the luminal surface of the artery. The thickening of the inner lining of the junction will eventually narrow the artery and cause thrombosis of the junction. As a result, the endograft will fail.

### SUMMARY OF THE INVENTION

It is an object of the present invention to minimize the flow changes that occur at the end sections of an endograft. This is achieved by varying the compliance in different sections of the endograft.

In one configuration, the endograft includes one or more struts that are divided into two or more portions. These portions are distinguished from each other by having different compliances. Some configurations may have a transition of compliance between the portions.

In one configuration, the endograft is divided into three portions, a proximal end portion, a distal end portion, and a central portion. In this configuration, the proximal and distal end portions have a higher compliance than the central portion.

In another configuration, the endograft is divided into two portion, a proximal portion and a distal portion. These portions have different compliances.

The difference in compliance can be achieved, for example, by using wire struts of different gauges, varying the spacing of the struts in the different portions, varying the geometry of the struts, using struts of different alloys, or using struts of differing elasticity.

### BRIEF DESCRIPTION OF THE DRAWINGS

The present invention will be illustrated with reference to the figures. Such figures are intended to be illustrative rather than limiting. They are included to facilitate the explanation of the configurations of the present invention. The figures are for illustrative purposes only, are not drawn to scale, and are not intended to serve as engineering drawings.

FIG. 1 is a longitudinal cross section view of the endograft having three portions.

FIG. 2 is a longitudinal cross section view of the endograft having five portions.

FIG. 3 is a longitudinal cross section view of the endograft having two portions.

### DETAILED DESCRIPTION OF THE INVENTION

The present invention will next be illustrated with reference to the figures. Such figures are intended to be illustrative rather than limiting and are included herewith to facilitate the explanation of exemplary features of configurations of the present invention. Unless otherwise noted, the figures are not to scale, and are not intended to serve as engineering drawings.

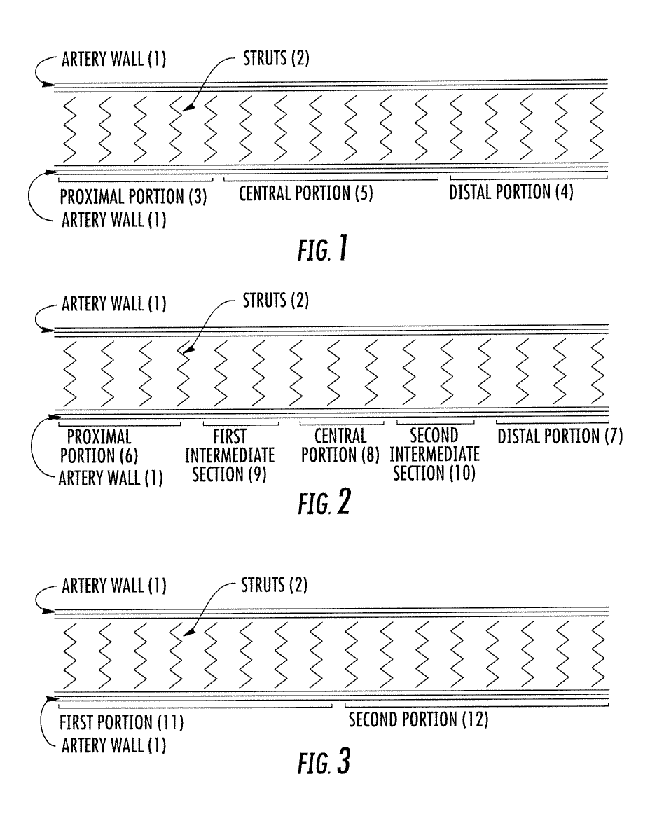

Referring now to the drawings, FIG. 1 shows a cross sectional view of an endograft according to an aspect of the present invention. The endograft is shown attached to the arterial wall 1. The endograft is preferably a tubular structure comprised of one or more struts 2 which are attached to a graft sheath made of polyester, Dacron, or polytetrafluoroethylene (PTFE). In this configuration, the endograft is divided into three portions: a proximal portion 3, a distal portion 4, and a central portion 5 located between the proximal portion 3 and the distal portion 4. The proximal portion 3 is located closer to the aorta and the heart than the distal portion 4.

In this configuration, the proximal portion 3 and the distal portion 4 have a different compliance than the central portion 5. Compliance refers to the dimensional change (diameter) that follows an intraluminal pressure change. It is traditionally defined as C=ΔD/DP_{P} where D is diameter in diastole, ΔD is the change in diameter and P_{P} is the pulse pressure. In a preferred configuration, the proximal portion 3 and the distal portion 4 have a compliance which is greater than the compliance of the central portion 5. In a preferred configuration the length of the proximal portion 3 is no greater than four times the diameter of the endograft and the length of the distal portion 4 is no greater than four times the diameter of the endograft. It is desirable for the proximal portion 3 and distal portion 4 to have a higher compliance than the central portion 5 because this will avoid the brusque flow changes and energy losses that result from an abrupt change in compliance between the artery and the endograft.

Some configurations may have a transition in compliance between the proximal portion 3 and the central portion 5 and a transition in compliance between the distal portion 4 and the central portion 5 such that there is an area of intermediate compliance.

FIG. 2 shows a cross sectional view of an endograft according to a further aspect of the present invention. As shown in FIG. 2, the endograft includes one or more struts 2, and is divided into five portions: a proximal portion 6, a distal portion 7, a central portion 8, a first intermediate section 9 located between the proximal portion 6 and the central portion 8, and a second intermediate section 10 located between the distal portion 6 and the central portion 8. In this configuration, the first intermediate section 9 and the second intermediate section 10 have a compliance which is different from the proximal portion 6, the distal portion 7, and the central portion 8. Preferably, the proximal portion 6 and the distal portion 7 have the highest compliance values, the central portion 8 has the lowest compliance value, and the first intermediate section 9 and the second intermediate section 10 have compliance values which are lower than the proximal portion 6 and distal portion 7 but higher than the central portion 8.

Referring now to FIG. 3 which shows a cross sectional view of an endograft according to yet a further aspect of the present invention is shown. As shown in FIG. 3, the endograft includes of one or more struts 2, and is divided into two portions: a first portion 11 and a second portion 12. In this configuration, the first portion 11 has a different compliance than the second portion 12. Preferably, the compliance of the first portion 11 is higher than the compliance of the second portion 12.

The difference in compliance in each configuration can be achieved, for example, by using struts which are of different gauges. For example, the struts in the first portion 11 are of a lower gauge than the struts in the second portion 12.

The difference in compliance can also be achieved by varying the spacing of the struts in the different portions. For example, the struts in the first portion 11 can be spaced further apart than the struts in the second portion 12.

Additionally, the difference in compliance can be achieved by varying the geometry of the struts. For example, the struts in the first portion 11 can have a geometry that has a higher compliance than the struts in the second portion 12.

Further, the difference in compliance can be achieved by using struts of different alloys. For example, the struts in the first portion 11 can be comprised of an alloy with a higher compliance than the struts in the second portion 12. Examples of alloys which can be used include Nitinol, Elgiloy, Tantalum, and stainless steel.

Also, the difference in compliance can be achieved by using struts of differing elasticity. For example, the struts in the first portion 11 have a higher elasticity than the struts in the second portion 12.

Although the present invention has been described in relation to particular configurations thereof, many other variations and modifications will become apparent to those skilled in the art. Therefore, the present invention should not be limited by the specific disclosure herein, but only by the appended claims.

What is claimed is:

1. A tubular endograft, comprising:
a tubular structure having a proximal portion, a distal portion, and a central portion located between the proximal portion and the distal portion,
the proximal portion and the distal portion having a different compliance than the central portion.

2. The tubular endograft of claim 1, wherein the compliance of the proximal portion and the distal portion is greater than the compliance of the central portion.

3. The tubular endograft of claim 2, wherein there is a transition in compliance between the proximal portion and the central portion and there is a transition in compliance between the central portion and the distal portion.

4. The tubular endograft of claim 1, further comprising:
a first intermediate section interposed between the proximal portion and the central portion, the first intermediate portion having a compliance different from that of the proximal portion and the central portion; and
a second intermediate section interposed between the central portion and the distal portion, the second intermediate portion having a compliance different from that of the distal portion and the central portion.

5. The tubular endograft of claim 1, wherein each of the proximal portion, the distal portion and the central portion comprise one or more struts.

6. The tubular endograft of claim 5, wherein the one or more struts in the proximal portion and the one or more struts in the distal portion have a lower gauge than the one or more struts in the central portion.

7. The tubular endograft of claim 5, wherein a spacing of the one or more struts in the central portion is different than a spacing of the one or more struts in the proximal portion and the one or more struts in the distal portion.

8. The tubular endograft of claim 7, wherein the one or more struts in the proximal portion and the distal portion are further apart than the one or more struts in the central portion.

9. The tubular endograft of claim 5, wherein a geometry of the one or more struts in the central portion is different than a geometry of the one or more struts in the proximal portion and the one or more struts in the distal portion.

10. The tubular endograft of claim 5, wherein the one or more struts in the proximal portion and the one or more struts in the distal portion are comprised of an alloy having a higher compliance than the one or more struts in the central portion.

11. The tubular endograft of claim 5, wherein an elasticity of the one or more struts in the proximal portion and the one or more struts in the distal portion is higher than an elasticity of the one or more struts in the central portion.

12. The tubular endograft of claim 1, wherein a length of the proximal portion is not greater than four times a diameter of the endograft and a length of the distal portion is not greater than four times the diameter of the endograft.

13. A tubular endograft, comprising:
a tubular structure having a first portion and a second portion,
the first portion having a different compliance than the second portion.

14. The tubular endograft of claim 13, wherein the compliance of the first portion is greater than the compliance of the second portion.

16. The tubular endograft of claim 13, further comprising an intermediate section interposed between the first portion and the second portion, the intermediate section having a compliance different from that of the first portion and the second portion.

17. The tubular endograft of claim 13, wherein each of the first portion and the second portion comprise one or more struts.

18. The tubular endograft of claim 17, wherein the one or more struts in the first portion have a lower gauge than the one or more struts in the second portion.

19. The tubular endograft of claim 17, wherein a spacing of the one or more struts in the first portion is different than a spacing of the one or more struts in the second portion.

20. The tubular endograft of claim 19, wherein the one or more struts in the first portion are further apart than the one or more struts in the second portion.

21. The tubular endograft of claim 17, wherein a geometry of the one or more struts in the first portion is different than a geometry of the one or more struts in the second portion.

22. The tubular endograft of claim 21, wherein the one or more struts in the first portion are comprised of an alloy having a higher compliance than the one or more struts in the second portion.

23. The tubular endograft of claim 17, wherein an elasticity of the one or more struts in the first portion is higher than an elasticity of the one or more struts in the second portion.
